(19) United States
(12) Patent Application Publication
(10) Pub. No.: US 2010/0209484 A1
(43) Pub. Date: Aug. 19, 2010

(54) Transdermal Triptan Delivery System

(76) Inventors: Hoo-Kyun Choi, Dong-gu (KR); Je Phil Ryoo, Princeton, NJ (US)

Correspondence Address: GIBBONS P.C., ONE GATEWAY CENTER, NEWARK, NJ, 07102, US

(21) Appl. No.: 12/704,673

(22) Filed: Feb. 12, 2010

Related U.S. Application Data
(60) Provisional application No. 61/207,852, filed on Feb. 13, 2009.

(51) Int. Cl. A61K 9/70; A61K 31/422; A61P 25/06

(52) U.S. Cl. 424/449; 514/376

(57) ABSTRACT

This invention relates to transdermal system for the systemic administration of an active ingredient such as a triptan selected from the group comprising eletriptan, frovatriptan, sumatriptan, zolmitriptan, naratriptan, rizatriptan and almotriptan uniformly over periods of 12 to 24 hours or more thereby reducing the probability of recurring migraine. The formulation includes active ingredient, adhesive matrix and permeation enhancer. The invention also relates to storage stable formulations and methods for preventing migraine and cessation of an ongoing migraine event.

### CROSS-REFERENCE TO RELATED APPLICATIONS

This application claims priority of U.S. Provisional Patent Application Ser. No. 61/207,852, entitled “Transdermal Triptan Delivery System,” filed on Feb. 13, 2009, which is incorporated by reference herein.

### FIELD OF THE INVENTION

The present invention relates to new pharmaceutical formulations, their preparation and use in treatment of disease. In particular, the present invention relates to pharmaceutical formulation of the anti-migraine drugs in the class of triptans. There are seven triptans: eletriptan, frovatriptan, sumatriptan, zolmitriptan, naratriptan, rizatriptan and almotriptan for transdermal application. The invention also relates to storage stable formulations and methods for preventing migraine and cessation of an ongoing migraine event.

### BACKGROUND OF THE INVENTION

The present invention relates to a transdermal patch for administering triptans. More particularly, the invention concerns transdermal delivery of triptans from a patch that can deliver a therapeutically effective dose of a triptan through the skin of a patient in need of treatment with a triptan for an extended period of time of at least 24 hours or more.

Triptans are selective 5HT₁-receptor agonists. Triptans are medicines for the treatment of migraine headaches. There are seven triptans: eletriptan, frovatriptan, sumatriptan, zolmitriptan, naratriptan, rizatriptan and almotriptan. Triptans are specific and selective agonists for the 5-HT₁ receptors. Sumatriptan binds to 5-HT_{1D} receptors, zolmitriptan, rizatripan, naratripan, almotriptan and frovatriptan bind to 5-HT_{1B/1D} and eletriptan binds to 5-HT_{1B/1D/1F} receptors. Triptans supposed mechanism of action are vasoconstriction effect on carotid arterial circulation without affecting cerebral blood flow, peripheral neuronal inhibition and inhibition of transmission through second order neurons of the trigeminocervical complex.

Zolmitriptan, has the chemical name (S)-4-{{3-[2-(dimethylaminoethyl)-1H-indol-5-yl]methyl}-2-oxazolidinone. The 5HT1-receptor mediates vasoconstriction and thus modifies blood flow to the carotid vascular bed. 5HT1-receptor agonists are beneficial in the prophylaxis and treatment of disease conditions wherein vasoconstriction in the carotid vascular bed is indicated, for example migraine, cluster headache and headache associated with vascular disorders, hereinafter referred to collectively as “migraine” or “migraine conditions”. Triptans have been developed for the acute treatment of migraine in the form of tablets for eletriptan, frovatriptan, sumatriptan, zolmitriptan, naratriptan, rizatriptan and almotriptan. These products are intended to be taken in amounts up to a maximum of 5 to 200 mg per day. Table 1 summarizes current dosing regimens for triptan products.

TABLE 1
Triptan Dosing Regimens

| | | | | Maximum 24 | |
|---|---|---|---|---|---|
| Generic Name | Dose | | Brand Name | hour Dose | |
| Almotriptan | 12.5 | mg | Axert | 12.5 | mg |
| Eletriptan | 40 | mg | Relpax | 80 | mg |
| Frovatriptan | 2.5 | mg | Frova | 7.5 | mg |
| Naratriptan | 2.5 | mg | Amerge | 5.0 | mg |
| Rizatriptan | 10 | mg | Maxalt | 30 | mg |
| Sumatriptan | 100 | mg | Imitrex | 200 | mg |
| Sumatriptan | 6 | mg | Imitrex | 6 | mg |
| injection | | | | | |
| Sumatriptan | 20 | mg | Imitrex | 40 | mg |
| nasal spray | | | | | |
| Zolmitriptan | 2.5 | mg | Zomig | 10 | mg |
| Zolmitriptan | 5 | mg | Zomig ZMT | 10 | mg |
| Zolmitriptan | 5 | mg | Zomig ZMT | 10 | mg |

Oral form unless otherwise noted.

U.S. Pat. No. 5,466,699 discloses a class of chemical compounds, indolyl compounds for the treatment and prophylaxis of migraine. This class of compounds, also known as triptans, may be formulated for oral, sublingual, buccal, parenteral (for example subcutaneous, intramuscular or intravenous), rectal, topical and intranasal administration.

Triptans are generally well tolerated, with most adverse events being mild-to-moderate, transient and resolving without intervention or the need for treatment withdrawal. However, orally delivered triptan drugs produce gastrointestinal disturbances. Thus there is an interest in developing alternative modes of delivering triptans which do not have the gastrointestinal side effects produced by oral formulations but which are more convenient than injection methods. Many patients have a recurrent migraine later in the day, and only one such recurrence in a day can be treated with a second dose of triptan. Using a transdermal patch as the second dose, releasing triptan over an extended period of 12 to 24 or more hours would greatly relieve recurrent migraine.

Certain triptans are available in alternate dosage forms. Sumatriptan is available in a nasal spray and injectable dosage form. Zolmitriptan is available in a nasal spray.

Transdermal devices for the delivery of biologically active agents have been used for maintaining health and therapeutically treating a wide variety of ailments. For example, analgesics, steroids, etc., have been delivered with such devices. Such transdermal devices include patches in which a biologically active agent is delivered to the body tissue passively without use of an additional energy source. Many such devices have been described, for example, U.S. Pat. No. 3,598,122 describes a bandage for use in the continuous administration of systemically active drugs by absorption through the skin or oral mucosa. The bandage has a backing member having on one surface a reservoir containing a systemically active drug. The reservoir has a wall distant from the backing member and permeable to passage of the drug. A pressure sensitive adhesives layer, also permeable to the drug adapted for contact with the skin or mucosa of a patient. U.S. Pat. No. 4,379,454 describes a unit dosage form and method that co-administer a drug and a percutaneous absorption enhancer to a predetermined area of skin. The drug is administered to the skin at a rate at least as great as the rate at which the skin is capable of absorbing it while the percutaneous absorption enhancer is administered at a substantially constant rate that increases the permeability of the treated area of skin to the drug to a magnitude such that sufficient drug is absorbed to provide a therapeutically effective level of drug in the bloodstream of the individual or patient. Accordingly the rate of drug administration is controlled by the rate at which the skin absorbs the drug whereas the rate of percutaneous absorption enhancer administration is controlled by the rate at which the enhancer is released from the dosage form to the skin surface. The rate of drug administration is, therefore, controlled indirectly by the controlled co-release of the enhancer since the latter affects the rate at which the skin will absorb the drug. U.S. Pat. No. 4,568,343, describes a method for enhancing the transdermal flux of a transdermally deliverable drug through intact skin in which the drug is delivered simultaneously with polyethylene glycol monolaurate. Preferred embodiments of transdermal therapeutic systems for delivering drug and polyethylene glycol monolaurate employ matrix containing drug at a concentration above saturation.

Anderson, et al., WO/2007/120747, describe transdermal methods and systems for the delivery of anti-migraine compounds using iontophoretic patches. Specifically, Anderson, et al. describe a device for the delivery of anti-migraine drugs, particularly triptan serotonin agonists including sumatriptan and naratriptan. In this delivery method, ions bearing positive charge are driven across the skin at the site of an electrolytic electrical system anode while ions bearing negative charge are driven across the skin at the site of an electrolytic system cathode.

The use of transdermal patches for drug delivery is particularly beneficial when it is desired to maintain a constant blood level of drug in the patient for extended periods of time. There is an added benefit in that oftentimes, the required dose of a drug when delivered by a 24 hour transdermal patch can be one half or less that of the dose delivered by a single once a day intravenous or oral dose. When taken orally, triptans suffer significant reduction in bioavailability due to loses in the digestive system. The following have been reported as the bioavailable amount after oral ingestion: Sumatriptan 14%; Zolmitriptan 40%; Naratriptan 63% in males—74% in females; Rizatriptan 47%; Almotriptan 69%; Eletriptan 50% and Frovatriptan 24% in males—30% in females. By avoiding the digestive system, essentially 100% of the absorbed triptan is bioavailable.

Hence, a transdermal product of a triptan, with a sustained action, and not requiring electrodes and sophisticated electronic equipment would be valuable in providing clinical benefit of prolonged, pain-free response to patients suffering from migraine.

### BRIEF SUMMARY OF THE INVENTION

The invention provides a self adhesive, transdermal triptan delivery device and formulations that deliver triptan at a therapeutically effective level. The formulations have no significant irritation potential and contain sufficient drug and enhancer to support 24 hour or more hours delivery at a reasonable adhesive layer thickness. The preferred formulation is a patch. The patch is comprised of a backing layer and an adhesive matrix containing the triptan.

The present invention provides a triptan transdermal patch wherein the triptan is selected from the group comprising eletriptan, frovatriptan, sumatriptan, zolmitriptan, naratriptan, rizatriptan and almotriptan or pharmaceutically acceptable salts thereof. Suitable pharmaceutically acceptable salts of the compounds of this invention include acid addition salts which may, for example, be formed by mixing a solution of the pharmaceutically active agent according to the invention with a solution of a pharmaceutically acceptable non-toxic acid such as hydrochloric acid, fumaric acid, maleic acid, succinic acid, acetic acid, benzoic acid, oxalic acid, citric acid, tartaric acid, carbonic acid or phosphoric acid. These anti-migraine agents are present in the dosage form in an amount effective for combating migraine by either treating the migraine headache and related symptoms or preventing the onset of the migraine headache and related symptoms, wherein the active surface area of the patch is about 5 cm² to about 140 cm² in active surface area.

The incorporation of the ingredients, such as one or more triptans and/or permeation enhancers, of the formulations provides enhanced rheological properties suitable for transdermal delivery. Further, the present invention identifies acrylate polymers that can dissolve such enhancers and triptan to facilitate the required solubility and flux for transdermal delivery. The matrix of this invention may further comprise a primary absorption enhancer selected from the group consisting of cineole and terpenes. Preferred terpenes include limonene, cymene, pinene, pellandrene and the like.

In one aspect, a transdermal delivery device is provided with high enough zolmitriptan content, preferably completely dissolved into a drug reservoir matrix wherein the matrix is an acrylate polymeric material with desirable rheological properties.

In one aspect, the present invention provides a self adhesive system for transdermal delivery of a triptan.

In another aspect, the present invention provides a transdermal triptan delivery system with little or no cold flow, and resulting in adequate on tack and adhesion for applying to a body surface.

In one aspect, a triptan according to this invention penetrates the skin at therapeutically effective rates without requiring additional permeation enhancers, though permeation enhancers may be utilized to increase the flux.

Another aspect of the invention is to provide a triptan transdermal patch that is effective in treating or preventing migraine in a patient for periods of 12 to 24 hours or more.

It is a aspect of this invention to provide a transdermal patch that can deliver zolmitriptan at a rate that attains a therapeutic level to avoid or minimize migraine in a patient with less than 140 cm² in active surface area.

It is another aspect of the invention to provide a transdermal zolmitriptan patch that avoids or minimizes skin irritation.

Another aspect of the invention is to provide a transdermal zolmitriptan patch that is effective in treating migraine for periods of 24 hours or more.

In one aspect of the invention, the present invention further provides a transdermal patch that can deliver a triptan at a rate that attains a therapeutic level for the prevention of migraine and cessation of an ongoing migraine event in an individual or patient in need so such prevention and cessation without the use of electrolytic charge systems.

These and other features of the invention will be apparent to those skilled in the art from the following detailed description and appended claims when taken in conjunction with the examples and figures.

### BRIEF DESCRIPTION OF THE DRAWINGS

FIG. 1 Shows permeation of zolmitriptan (4 weight % of the adhesive matrix) from various acrylic adhesive matrixes.

FIG. 2 Shows permeation of zolmitriptan (4 weight % of the adhesive matrix) from various acrylic adhesive matrixes containing hydroxyl functional group.

FIG. 3 Effect of drug loading in 87-2510 matrix.

FIG. 4 Effect of adhesive matrix thickness at 4% drug loading.

FIG. 5 Effect of polyoxyethylene (2) cetyl ether concentration on the permeation of zolmitriptan, in presence of ethyl cellulose and povidone K-30

FIG. 6 Effect of lauryl alcohol on the permeation of zolmitriptan

FIG. 7 Effect of limonene and polyglyceryl-3 oleate, at different drug concentrations, on the permeation of zolmitriptan.

FIG. 8 Effect of limonene and cineole on the permeation of zolmitriptan.

FIG. 9 Penetration rate versus time for aged patches.

FIG. 10 Penetration rate versus time for aged patches.

FIG. 11 Effect of Sucrose ester P 1670 and cineole on the permeation of zolmitriptan.

### DETAILED DESCRIPTION OF THE INVENTION

To date, successful therapeutically effective transdermal delivery of triptans has not been reported. The applicants have discovered a patch composition and method to deliver a therapeutically effective dose of a triptan. The bioavailablilty of oral doses for various triptans has been reported to be: Sumatriptan 14%; Zolmitriptan 40%; Naratriptan 63% in males—74% in females; Rizatriptan 47%; Almotriptan 69%; Eletriptan 50% and Frovatriptan 24% in males—30% in females. Oral administration of triptans suffers from poor bioavailability, partly due to presystemic metabolism. Some of the triptan gets broken down in the stomach before it reaches the target arteries. The patch delivery system avoids this presystemic metabolism as would the injection and nasal spray delivery systems.

Based on these estimates and on reported data from the injection and nasal spray dosages available for some of the triptan compounds, in order to achieve therapeutically effective blood levels in humans, about 2 to about 4 mg per day of zolmitriptan should be delivered transdermally, about 7.5 mg per day of Almotriptan should be delivered transdermally, about 1.0 to about 3.0 mg per day of frovatriptan should be delivered transdermally, about 1 to about 2.0 mg per day of naratriptan should be delivered transdermally, about 4 to about 12.0 mg per day of rizatriptan should be delivered transdermally, about 5 to about 20.0 mg per day of sumatriptan, should be delivered transdermally. The terms human and patient are used herein as being equivalent. The above estimates are based on delivery of the free base content of each triptan compound. The triptan is selected from the group comprising eletriptan, frovatriptan, sumatriptan, zolmitriptan, naratriptan, rizatriptan and almotriptan or pharmaceutically acceptable salts thereof. Suitable pharmaceutically acceptable salts of the compounds of this invention include acid addition salts which may, for example, be formed by mixing a solution of the pharmaceutically active agent according to the invention with a solution of a pharmaceutically acceptable non-toxic acid such as hydrochloric acid, fumaric acid, maleic acid, succinic acid, acetic acid, benzoic acid, oxalic acid, citric acid, tartaric acid, carbonic acid or phosphoric acid.

The zolmitriptan transdermal patch formulations of the present invention result in a hairless mouse skin flux rate of at least about 10 μg/cm² hr. The hairless mouse skin is more permeable than human skin to some of the drugs (Robert L. Bronaugh, Raymond F. Stewart, Elaine R. Congdon, Methods for in vitro percutaneous absorption studies II. Aminal models for human skin, Tox. And Applied Pharmacology. 62, 1982, 481-488). Even if we assume that the hairless mouse skin is 4 times more permeable than human skin, less than 40 cm² area is required to deliver 2 mg of zolmitriptan per day. Thus the patches of the present invention are therapeutically useful at active surface areas of less about 5 cm² to about 140 cm² for extended time of period. Preferably, the patch will have an active surface area of about 5 cm² to about 100 cm² and more preferably about 5 cm² to about 80 cm² and most preferably about 10 cm² to about 60 cm². As used herein, the term ‘active surface area’ refers to the area of the patch where drug is transmitted to the skin. Accordingly inactive surface may, for example be peripheral to the active surface area and may provide an adhesive or structural function. Preferably, the patch's active surface area, the area in contact with the patient's skin, is also the adhesive surface area.

Triptans have varying degrees of lipophilic character as follows: Sumatriptan Low; Zolmitriptan Moderate; Naratriptan High; Rizatriptan Moderate; Almotriptan; —Eletriptan High; Frovatriptan Low. Zolmitriptan, for example, is soluble in water and methanol. It has a pKa of 9.64 and molecular weight of 287.36. The compound is white to almost white, crystalline material that melts around 136° C. As such, it is a relatively difficult drug to deliver transdermally because of the extremely lipophilic barrier of the stratum corneum. For adequate skin penetration rates, a chemical enhancer may be necessary. As used herein, the term ‘enhancer’ is meant to encompass any compound or combination of compounds that increases the permeability of zolmitriptan to the stratum corneum. As explained in the literature (Lisa M. Russell, Sandra Wiedersberg, M. Begoña Delgado-Charro, The determination of stratum corneum thickness An alternative approach, Eu. Journ. Pharm. Biopharm. 69, 2008, 861-870), the stratum corneum layer is only 10-15 microns thick. However, it is the principal barrier to transdermal permeation. Depending upon the type, some enhancers migrate into the upper stratum corneum layers of the skin and remain there for a prolonged period of time. The enhancers that migrate into the skin serve to increase the mobility and solubility of triptans, for example, zolmitriptan, into the skin. Other types of enhancers are capable of increasing the permeability of drugs to the stratum corneum. The size of the patch is determined by the transdermal percutaneous absorption rate, referred to herein as flux (μg/cm² hr) of the triptan and the therapeutically effective dose or blood level.

Patch Design

Several types of transdermal patches can be used for the delivery of triptans. The simplest patch design is an adhesive matrix type and consists only of a backing material at the skin-distal side of the patch and a drug depot layer comprised of drug, adhesive and permeation enhancer when desired that attaches directly to a patient's skin. In this patch it is possible to incorporate lipophilic permeation enhancers, such as a terpene selected from the group comprising: cineole, limonene, cymene, pinene, and pellandrene and or non-terpene enhancers selected from the group consisting of polyoxyethylene alkyl ethers such as polyoxyethylene cetyl and lauryl ethers, fatty alcohols such as, lauryl alcohol, glycerin fatty acid ester, fatty acid alkyl esters and their derivatives, sucrose fatty acid esters, polyglyceryl-3 oleate, isopropyl myristate and isopropyl palmitate. The basic embodiment of the present invention comprises an impermeable backing layer and an adhesive matrix layer which serves both as a depot for a triptan and a means of adhering the device to the skin.

As described above in general triptan case, several types of transdermal patches can be used for the delivery of zolmitriptan. The simplest patch design is an adhesive matrix type and consists only of a backing material at the skin-distal side of the patch and a drug depot layer comprised of enhancer, drug and adhesive that attaches directly to a patient's skin.

The basic embodiment of the present invention comprises an impermeable backing layer and an adhesive matrix layer which serves both as a depot for zolmitriptan and a means of adhering the device to the skin. This embodiment is generally referred to as a “patch.”

The impermeable backing layer defines the non-skin facing side of the patch in use. The functions of the backing layer are to provide an occlusive layer that prevents loss of the triptan and the enhancers, in particular solvent enhancer, to the environment and to protect the patch. The material chosen should exhibit minimal triptan and enhancer permeability. The backing layer can be clear or translucent because triptans are not photosensitive. Ideally, the backing material should be capable of forming a support onto which the triptan containing mixture can be cast and to which it will bond securely. As an alternative to casting the matrix directly on the backing, the polymer matrix may be cast separately and later stuck to the backing material.

Compositions, transdermal formulation, comprising a triptan and a pressure sensitive adhesive in accordance with this invention typically also comprise a backing membrane and a release liner, each of which can comprise materials conventionally used in transdermal drug delivery compositions. The backing is attached to one face of the adhesive matrix; the release liner releasably covers the adhesive matrix formulation attached to a second, opposing face of the adhesive matrix.

The backing can comprise any material conventionally used as such in transdermal patch compositions. The material chosen for the backing is one which is flexible and impermeable to the drug, and, if desired, can be colored or labeled. The backing provides support and a protective covering for the dosage unit. Suitable backing materials include those known in the art for use with pressure sensitive adhesives. For example, the backing can comprise a polyolefin, polyether, multi-layer EVA film, polyester, polyurethane or combination thereof. Preferred backings include Scotch Pak 9732, a polyester film laminate supplied by 3M, MEDIFLEX® 1000, a polyolefin manufactured by Mylan Technologies, Inc. or Volara® 6 EO 0.031 SK foam, a cross-linked fine-celled foam manufactured by Votek, Inc.

Release liners are used to cover the surface of the pressure-sensitive adhesive during storage. The release liner can prevent evaporative loss of one or more components of the drug delivery system or matrix. The release liner is removed and discarded from the composition to expose the adhesive matrix which will be applied to the patient's skin. Suitable release liners include those known in the art for use with pressure sensitive adhesive composition. For example, the release liner can comprise a fluorosilicone coated polyester, silicone coated polyester or a UV cured, silicone-coated polyester. Preferred release liners include MEDIRELEASE® 2500, MEDIRELEASE® 2249 and MEDIRELEASE® 2226, each manufactured by Mylan Technologies, Inc., Clearsil® release liner UV5A manufactured by CPFilms, Inc. or Scotchpak™ 1022, manufactured by 3M Pharmaceuticals/D.D.S. The release liner, however, can comprise other materials, including paper or paper-containing layers or laminates, various thermoplastics, polyester films, foil liners, and the like.

Non-adhesive components can be included in the adhesive composition, including preservatives, antioxidants and chelating agents. Suitable examples of such compounds include butylated hydroxyanisole (BHA), butylated hydroxytoluene (BHT), sodium metabisulfate, a tocopherol, maleic acid, ethylenediaminetetraacetic acid (EDTA), cysteine hydrochloride, colloidal silicone dioxide and metal oxides (ZnO, TiO.sub.2, etc.).

The triptan adhesive matrix layer comprises triptan dispersed in an adhesive polymer matrix. As used herein, the term ‘dispersed’ refers to the distribution of triptan throughout the matrix. The drug may be dispersed in a dissolved and/or undissolved state, but is preferably dissolved. In preferred embodiments, the content of triptan in the adhesive polymer (drug reservoir) comprises about 0.5 wt % to 20 wt % of the weight of the adhesive acrylate polymer; preferably about 0.5 wt % to 15 wt %; more preferable about 0.5 wt % to 10 wt %; more preferably about 1 wt % to 9 wt %. When the triptan is zolmitriptan, the content of zolmitriptan in the adhesive polymer is about 0.5 wt % to about 12 wt % of the weight of the adhesive polymer, preferably from about 0.5 wt % to about 10 wt %, more preferably from about 2 wt % to about 9 wt %, and most preferably from about 2 wt % to about 7 wt %.

In additional embodiments, the adhesive matrix layer may optionally contain permeation enhancers. Permeation enhancers may be used alone or in combination. Examples of permeation enhancers include terpenes wherein the terpene is selected from the group comprising: cineole, limonene, cymene, pinene, and pellandrene and non-terpenes such as polyoxyethylene alkyl ethers, fatty alcohols, glycerin fatty acid ester, fatty acid alkyl esters and their derivatives and sucrose fatty acid esters. Said enhancers include, but are not limited to polyoxyethylene cetyl and lauryl ethers; lauryl alcohol, limonene, cineole, polyglyceryl-3 oleate, isopropyl myristate and isopropyl palmitate. In preferred embodiments, the content of permeation enhancers in the drug reservoir comprises 0.1 wt % to 20 wt % of the weight of acrylate polymer; preferably 0.5 wt % to 15%; more preferably 1 wt % to 10%. When the permeation enhancer is a terpene, the terpene is present in an amount of from about 0.5 to about 20 percent of the weight of the adhesive polymer, preferably the terpene is present in an amount of from about 0.5 to about 15 percent of the weight of the adhesive polymer and more preferable the terpene is present in an amount of from about 1 to about 10 percent of the weight of the adhesive polymer.

The adhesive layer may be prepared as follows. First a solution of the adhesive polymer is obtained or prepared. Another solution of a triptan such as zolmitriptan and appropriately selected enhancers is prepared and mixed until the drug is dissolved or evenly dispersed. The triptan is dissolved in a suitable solvent such as but not limited to ethanol, ethyl acetate, isopropanol, propanol, methyl ethyl ketone and mixtures thereof. The triptan/enhancer solution is then added to the adhesive polymer solution and the mixture is homogeneously mixed. The solution is cast on the release liner coated with silicone. The casting is then left for the solvent to evaporate at room temperature or in an oven at an elevated temperature. After solvent evaporation, the adhesive matrix is laminated with a backing membrane.

The adhesive used in an adhesive matrix type patch can be selected from a variety of adhesives available commercially and known to those in the art. For example, common adhesives are those based on acrylic polymer. Acrylic polymers with carboxyl functional groups may not be used for the triptans due to interaction between amine group of the triptans and carboxyl group of the polymer. The selection of the adhesive is critical to realize a functioning adhesive matrix type transdermal patch. The enhancers and the drug are loaded directly into the adhesive and so the adhesive must retain its functioning properties in the presence of these additives. These adhesive properties include sufficient tack for good instantaneous adhesion to the skin as well as maintenance of adhesion. Often adhesives become stringy and gooey in the presence of the skin permeation enhancers leading to cohesive failure and residual adhesive left on the patient's skin after removal of the patch. In some cases, the patch looses adhesion altogether and falls off. The loss of tack and other adhesion properties generally dictates and limits the amount and type of enhancers that can be loaded into the adhesive matrix type patches. Some acrylate based adhesives, such as those available from National Adhesives, are able to withstand relatively high loadings of enhancers.

Acrylate adhesives are typically made by copolymerizing at least one acrylate or methacrylate monomer with at least one modifying monomer and at least one functional group-containing monomer. Exemplary acrylate and methacrylate monomers include 2-ethylhexyl acrylate, butyl acrylate, and isooctyl acrylate. Exemplary modifying monomers include vinyl acetate, ethyl acrylate, methacrylate, and methyl methacrylate. Exemplary functional groups include carboxyl and hydroxy groups such as those present on acrylic acid, methacrylic acid and hydroxy-containing monomers such as hydroxyethyl acrylate. Particularly suitable acrylate-based adhesives comprise polyacrylate adhesive copolymers such as those comprising a 2-ethylhexyl acrylate monomer. Suitable acrylate-based adhesives include those commercially available from the National Adhesives under the trademark Duro-Tak. These include, for example: Duro-Tak 87-900A, 87-9301, 87-4098 and 87-9088 acrylic non-curing pressure sensitive adhesives supplied in an organic solvent (ethyl acetate) all having no functional groups; Duro-Tak 87-202A, 387-2510/87-2510 acrylic non-curing pressure sensitive adhesives supplied in an organic solvent (ethyl acetate) all having —OH functional groups, 87-208A, 387-2287/87-2287, and 87-4287, acrylate-vinyl acetate non-curing pressure sensitive adhesives supplied in an organic solvent (ethyl acetate) solution all having —OH functional groups; and Duro-Tak 387-2516/87-2516 and 387-2525/87-2525 acrylate-vinyl acetate self-curing pressure sensitive adhesive supplied in an organic solvent solution all having —OH functional groups.

The thickness of the acrylate adhesive triptan containing matrix effects permeation of the triptan. The acrylate adhesive matrix has thickness of about 50 μm to about 200 μm, preferably about 60 μm to about 180 μm and more preferably about 75 μm to about 165 μm.

On application to the skin, the drug in the drug reservoir of the transdermal patch diffuses into the skin where it is absorbed into the bloodstream to produce a systemic therapeutic effect to relieve migraine or to prophylactically inhibit or prevent migraine conditions in the patient. Typically, it is preferable that a patient experiences an adequate effect within a few hours (e.g., 3-6 hours) of initial application. The surface area of the transdermal patch in contact with the skin is about 5 cm² to about 140 cm², preferably about 5 cm² to about 100 cm², more preferably about 10 cm² to about 60 cm². Patient plasma levels are about 0.5 ng/ml to about 4 ng/ml. The flux level using hairless mouse skin ranged from about 4 μg/cm² hr to about 26 μg/cm² hr. Flux levels are determined at about 12 to about 15 hours after patch application considering patch surface area. The transdermal patch is stable on storage. Preferably, the patch remains unchanged for about 6 months or longer wherein the patch when applied to a patient's skin it will deliver the triptan as therapeutically effective as a freshly made patch. More preferably, the patch remains unchanged for about 12 months or longer and most preferably, the patch remains unchanged for at least about 2 years, wherein the patch when applied to a patient's skin it will deliver the triptan as therapeutically effective as a freshly made patch. The term “unchanged” shall mean that the performance of the patch with respect to flux is within ±10% of the performance of an identical fresh patch and upon visual inspection of the patch, little or no cloudiness or color change is observed.

The onset of therapeutic action for tablet dosage forms of triptans is from about 30 minutes to about 60 minutes. Rapid onset of action is desired in a migraine treatment. The duration of action is short lasting about 3 to 6 hours. This leaves the patient subject to recurring migraine attacks later that same day. Only one such recurrence in a day can be treated with a second oral dose of triptan. Using a transdermal patch as the second dose, releasing triptan over an extended period of 12 to 24 or more hours would greatly relieve recurrent migraine. The method of treating would require that the patch be applied concomitantly to within about two hours of the patient taking an oral tablet or injectable dose of triptan. Preferably the patch would be applied within one hour of taking an oral tablet or injectable dose or triptan. The patch would begin to produce measurable blood levels of triptan from about 3 to 6 hours after application with maximum permeation flux commencing about 10 to 15 hours after application of the patch and continuing to about 24 hours or more. As can be seen in the examples, the beginning time of maximum flux is determined by the composition of the patch as is the level of the flux.

### EXAMPLES

The following examples are offered by way of illustration and not by way of limitation.

Measurement of Skin Permeation Rates

Skin permeation rates of various zolmitriptan/enhancer formulations were determined using flow through diffusion cells. Permeation experiments were done on isolated hairless mouse skin. A system comprising a multi channel peristaltic pump, a fraction collector, a circulating water bath and flow-through diffusion cells was used. Each flow-through cell had two arms, which allowed the receiver cell medium pumped to a fraction collector. The diffusion cell temperature was maintained at 37° C. by circulating water through the outer part of jacketed receiver cell. The surface area of receiver cell opening as 2 cm², and its volume was 5.5 ml Skin was excised from hairless mouse that was sacrificed with diethyl ether. Subcutaneous fat was removed with scissors and scalpel. Each of the flow-through diffusion cell components was connected via silicone rubber tubing with an internal diameter of 0.015 inches. The receiver cell was filled with a pH 6 buffer solution and the media stirred by Teflon-coated magnetic bar. The transdermal device was placed on the stratum corneum and the excised skin was mounted onto each receiver cell. And O-ring and cell top was placed on the top of each skin. These components are then clamped. The amount of drug permeated across the skin was calculated from the cumulative release. The samples were collected every 4 hours for 24 hours and assayed by HPLC. This method was used to measure skin permeation rates referred to as transdermal flux or flux in the examples.

Materials

Zolmitriptan was obtained from Gaobo Pharm-Chemicals (Beijing, China). Polyglyceryl-3 oleate (Plurol Olieque® CC497), Propylene glycol mono laurate (Lauroglycol®), and Polyoxy glycerate (Labrafil® 1944) were obtained from Gattefosse Korea (Seoul, South Korea). PEG sorbitan monooleate (Tween 80®), sorbitan monooleate (Span 80®), Propylene glycol (PG), Oleyl Alcohol® was purchased from Junsei Chemicals (Japan). Isopropyl palmitate (IPP), Isopropyl myristate (IPM), PEG-12 palm kernel glycerides (Crovol PK40®), and PEG-20 almond glycerides (Crovol A40®) were obtained from Croda (Parsippany, N.J., USA). (R)-(+) Lauryl alcohol, Limonene, Brij 52® and Brij 30® were purchased from Sigma Chemical (St. Louis, Mo., USA). Acrylic pressure sensitive adhesive solutions in organic solvents and polyisobutylene were obtained from National Starch and Chemical Company (Bridgewater, N.J., USA). Silicone pressure sensitive adhesive was obtained from Dow Corning (Midland, Mich., USA). All other chemicals were reagent grade or above and were used without further purification.

Analytical Method

Zolmitriptan was analyzed by an HPLC system (Shimadzu Scientific Instruments, MD), consisting of a UV detector (SPD-10A), reversed-phase C8 column, a pump (LC-10AD), and an automatic injector (SIL-10A). The method had been previously described (Vyas et. al., 2005). Briefly, the wavelength of the UV detector was 229 nm, the column temperature was maintained at 30° C., the flow rate was 1 ml/min and injection volume was 10 μl. The mobile phase used consisted of acetonitrile/phosphate buffer pH 7.5 (15/85).

Content Analysis

Drug content in patch was determined by extraction with methanol. Briefly, a patch sample of 4 cm² was put into bottle containing 100 ml methanol, sonicated for 15 mins and stirred with Teflon coated magnetic bar for 6 hours. The solution was filtered (Whatman 0 membrane, pore size 0.45 μm; NJ, USA) and analyzed by HPLC as described in the analytical method above.

Dissolution Study

Release profile of the patches was determined using paddle over disk method (Shah et. al., 1989). The patch was held in position by attaching it to a sinker at the bottom of dissolution flask. 500 ml of phosphate buffer (pH 6) was used as dissolution medium, temperature was set to 32° C. and paddle rpm of 50 provided the agitation. The size of the patch used in dissolution test was 15 cm².

Preparation of Zolmitriptan Adhesive Matrix Patches

All the formulations were prepared by the following procedure:

The drug solution was obtained by dissolving zolmitriptan in a suitable organic solvent such as ethanol, ethyl acetate, isopropanol, propanol, methyl ethyl ketone and mixtures thereof and permeation enhancer(s) were added. Adhesive solution and drug solution were mixed and stirred sufficiently. The mixture was cast on release liner coated with silicone and solvent was removed by evaporation. Then the dried adhesive layer was laminated onto the backing membrane. The drug and excipients are given as weight % of dry pressure sensitive adhesive polymer in all examples and tables.

### Example 1

### Effect of Adhesive Matrix

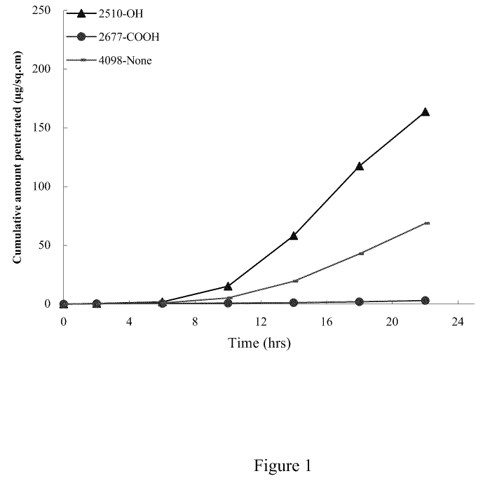

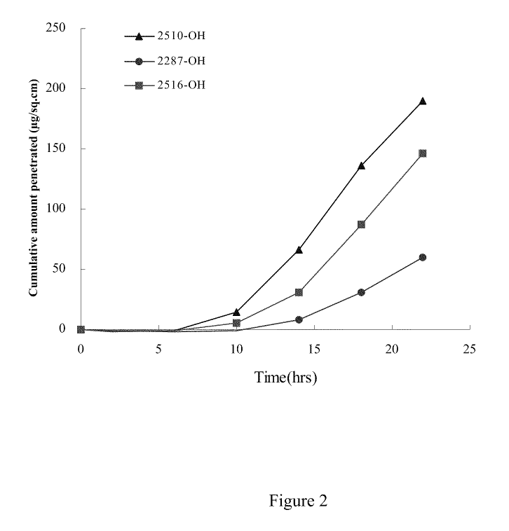

The effect of the pressure sensitive adhesive (PSA) matrix on the permeation of zolmitriptan was investigated using silicone, polyisobutylene (PIB) and acrylic adhesive matrix. Solubility of zolmitriptan was found to be inadequate in silicone and PIB adhesive matrixes. FIGS. 1 and 2 show permeation of zolmitriptan (4 weight % of the adhesive matrix) from various acrylic adhesive matrixes; National Adhesives Duro-Tak® 87-2510, 87-4098 and 87-2677. The permeation rate was lowest in the adhesive containing carboxyl functional group, National Adhesives Duro-Tak® 87-2677. This could be due to the interaction between amine group of zolmitriptan and carboxyl group of adhesive. Permeation rate of zolmitriptan in the acrylic adhesive matrix was highest with acrylic adhesive containing hydroxyl functional group, National Adhesives Duro-Tak® 87-2510. Further study on different kinds of acrylic adhesives containing hydroxyl functional group revealed that acrylic adhesive National Adhesives Duro-Tak® 87-2510 provided the best permeation characteristics of the acrylate adhesive polymers tested. (FIG. 2). The three acrylate adhesive polymers tested were National Adhesives Duro-Tak® 87-2510; 87-2287 and 87-2516. Only the acrylate adhesive polymers tested with hydroxyl (—OH) functional groups or no functional groups provided sufficient permeation characteristics. A comparison of the penetration flux and lag time for the acrylate adhesive matrices with differing functional groups is given in Table 2.

TABLE 2
The penetration rate and lag time for zolmitriptan
from different adhesive matrixes. (n = 3)

| Adhesive matrix | | Flux (μg/cm²hr) | Lag time (hrs) |
|---|---|---|---|
| without | | 6.16 | 10.7 |
| functional group | | | |
| with carboxyl | | 0.22 | NA* |
| functional group | 87-2510 | 15.6 | 9.3 |
| with hydroxyl- | 87-2287 | 6.5 | 13.0 |
| functional group | 87-2516 | 14.4 | 11.8 |

*NA = Not applicable

### Example 2

### Effect of Drug Concentration

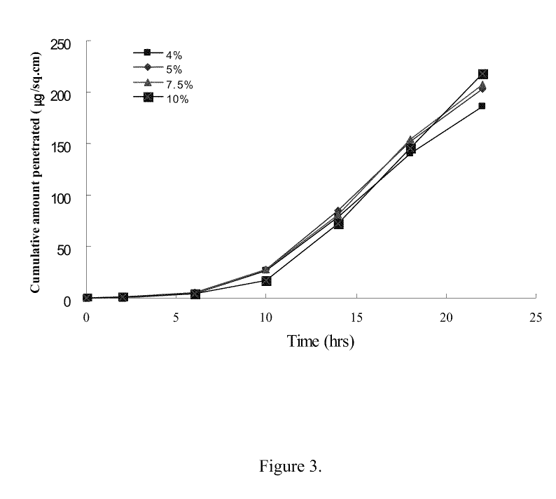

The effect of drug loading in 87-2510 matrix, on the amount of zolmitriptan permeated across hairless mouse skin as a function of time is shown in FIG. 3. The flux of zolmitriptan did not change significantly as the drug loading in matrix increased from 4% to 10% (weight % of the amount of polymer), indicating that saturation of zolmitriptan within the adhesive matrix was reached at ca. 4%. Patches with 4% drug load were clear as compared to the milky patches containing 5% or more drug load, and were used for further study.

TABLE 3
Effect of drug concentration on the penetration
rate and lag time for zolmitriptan. (n = 3)

| Concentration (Wt %) | Flux (μg/cm²hr) | Lag time (hrs) |
|---|---|---|
| 4 | 13.5 | 8.2 |
| 5 | 14.8 | 8.5 |
| 7.5 | 15.8 | 8.4 |
| 10 | 18.3 | 9.0 |

### Example 3

### Adhesive Matrix Thickness

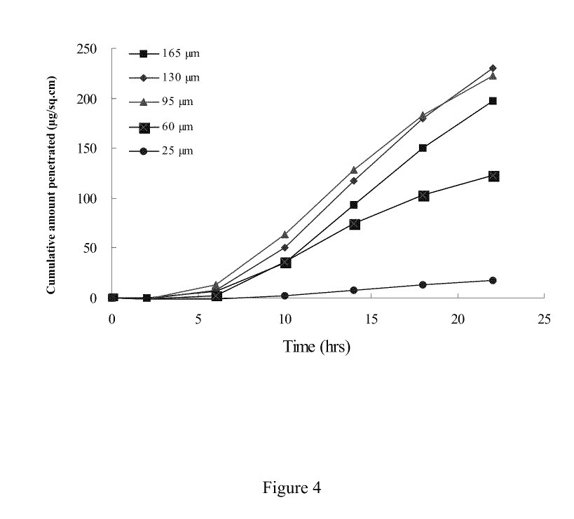

The effect of adhesive matrix thickness at 4% drug loading in National Adhesives Duro-Tak® 87-2510 matrix was investigated. FIG. 4 shows that the penetration rate of zolmitriptan increased when matrix thickness increased up to 95 μm and remained similar up to 130 μm. Further increase in the thickness resulted in lower permeation rate. The thickness of 100 μm was selected for further study.

TABLE 4
The penetration rate and lag time for zolmitriptan
from matrixes of different thickness. (n = 3)

| Matrix thickness (μm) | Flux (μg/cm²hr) | Lag time (hrs) |
|---|---|---|
| 165 | 13.0 | 7.4 |
| 130 | 14.1 | 7.0 |
| 95 | 11.7 | 5.2 |
| 60 | 6.1 | 5.5 |
| 25 | 1.2 | 9.0 |

### Example 4

Formulations (1 to 6) according to Table 5 were produced using acrylic adhesive with hydroxyl functional group (Durotak® 387-2516, National Adhesives) and methyl ethyl ketone as organic solvent for drug solution. Components are given in weight % of adhesive matrix.

TABLE 5
Effect of Permeation enhancers

| | Formulation | | | | | |
|---|---|---|---|---|---|---|
| | 1 | | | | | |
| | WT | 2 | 3 | 4 | 5 | 6 |
| Components | % | WT % | WT % | WT % | WT % | WT % |
| Zolmitriptan | 5 | 5 | 5 | 5 | 5 | 5 |
| Polyoxyethylene | | 5 | 7.5 | 10 | 12.5 | 15 |
| (2) cetyl ether | | | | | | |
| Ethyl cellulose | 2.5 | 2.5 | 2.5 | 2.5 | 2.5 | 2.5 |
| Povidone K-30 | 5 | 5 | 5 | 5 | 5 | 5 |
| Flux μg/cm²hr | 12.32 | 15.27 | 19.57 | 20.19 | 20.65 | 23.51 |

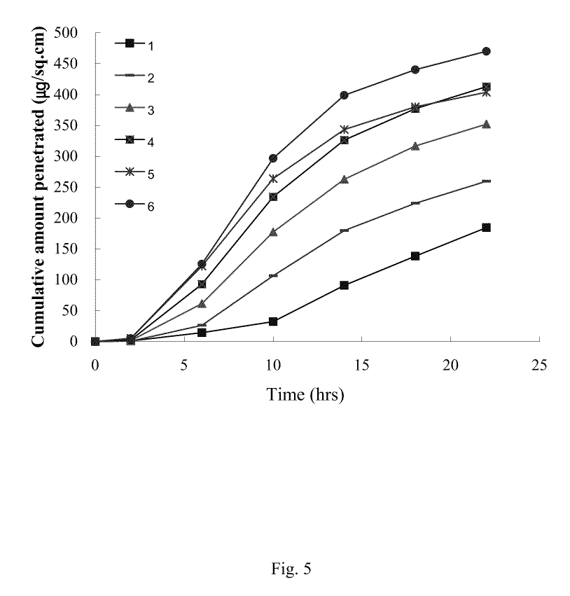

As clearly seen in FIG. 5, significant increase in the permeation rate could be obtained by using polyoxyethylene (2) cetyl ether as chemical enhancer. It should however be noted that even in the absence of enhancer, the flux obtained is sufficient to achieve therapeutic concentration of drug through reasonable size of the patch. Including ethyl cellulose and povidone K-30 in the ratio of 1:2, prevented crystallization of the formulation without impairing the permeation profile.

### Example 5

Formulations (7 to 10) according to Table 6 were prepared using acrylate adhesive, with hydroxy functional group, National Adhesives Duro-Tak® 387-2516 as the PSA and methyl ethyl ketone as solvent for drug solution.

TABLE 6

Formulation

| | 7 | 8 | 9 | 10 |
|---|---|---|---|---|
| Components | WT % | WT % | WT % | WT % |
| Zolmitriptan | 5 | 6 | 7 | 8 |
| Lauryl alcohol | 10 | 10 | 10 | 10 |
| Flux μg/cm²hr | 17.22 | 19.46 | 25.64 | 18.38 |

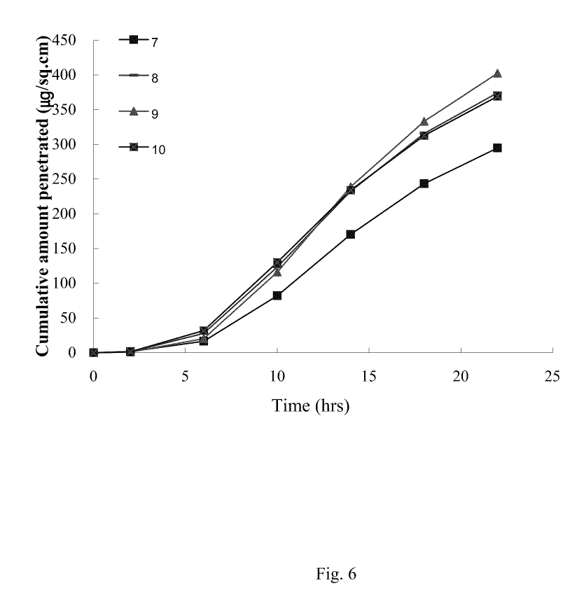

As is shown in FIG. 6, formulation 9 provided the highest flux. In the absence of enhancer, increasing the drug content did not provide higher flux.

### Example 6

Formulations (11 to 15) according to Table 7 were prepared using National Adhesives Duro-Tak® 387-2516 as the PSA and mixture of ethanol and ethyl acetate as solvent for drug solution.

TABLE 7
Polyglyceryl-3 oleate and limonene as enhancers.

| | Formulation | | | | |
|---|---|---|---|---|---|
| | 11 | 12 | 13 | 14 | 15 |
| Components | WT % | WT % | WT % | WT % | WT % |
| Zolmitriptan | 5 | 5 | 5.5 | 5.5 | 6 |
| Limonene | | 5 | | 5 | |
| Polyglyceryl-3 oleate | | 2 | 2 | 2 | 1 |
| Flux μg/cm²hr | 11.91 | 13.79 | 14.43 | 14.04 | 14.64 |

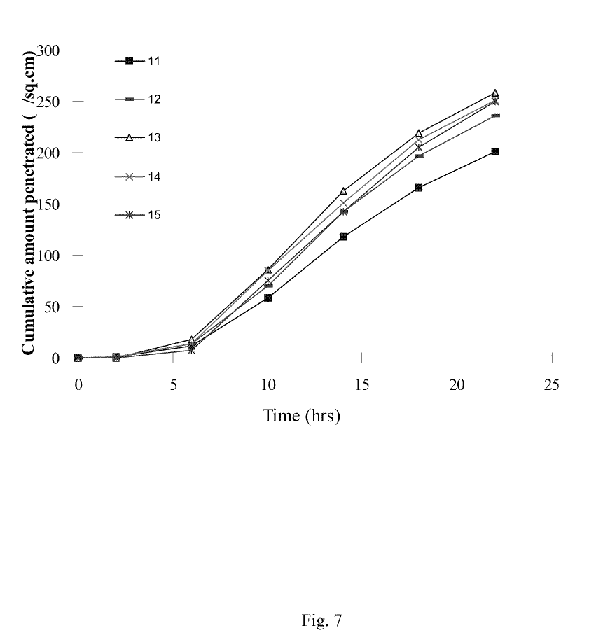

FIG. 7 shows the benefit of including polyglyceryl-3 oleate and limonene as enhancers in the transdermal formulation containing zolmitriptan.

### Example 7

Formulations (16 to 19) according to Table 8 were prepared using acrylate adhesive National Adhesives Duro-Tak® 387-2516 as the PSA and ethanol as solvent for drug solution.

TABLE 8

Formulation

| | 16 | 17 | 18 | 19 |
|---|---|---|---|---|
| Components | WT % | WT % | WT % | WT % |
| Zolmitriptan | 5.5 | 5.5 | 5.5 | 5.5 |
| Limonene | | 5 | | 2.5 |
| Cineole | | | 5 | 2.5 |
| Flux μg/cm²hr | 11.21 | 16.39 | 18.01 | 18.31 |

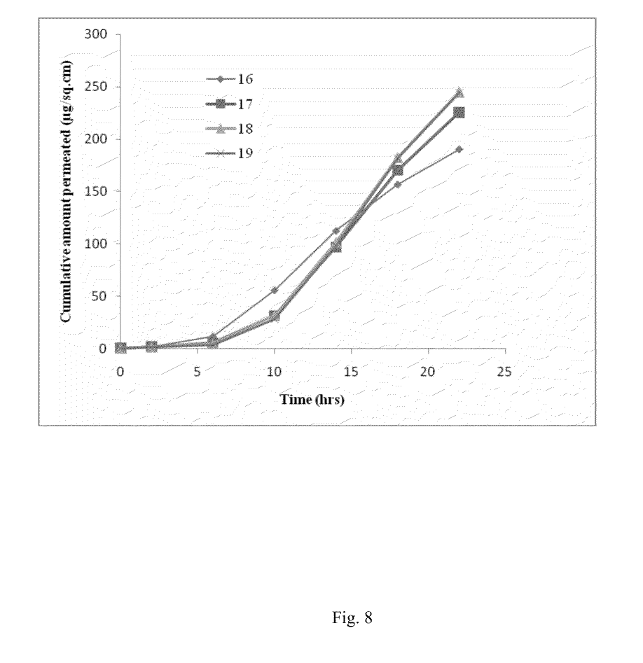

As is shown in FIG. 8, different combination of cineole and limonene were used as enhancers at the drug loading of 5.5% for crystal-free formulation.

### Example 8

### Physical Observation of Patch Formulation

Permeation is increased for a given concentration of triptan if the triptan is in solution in the matrix. The patches of Table 9 were stored at the room temperature for the designated period of time then observed by eye or/and a microscope. No crystals or white spots have been observed, and the patches are clear. The terpenes limonene and cineole have been found to act as permeation enhancers for triptans in the patch formulations while preventing the formation of crystals. This function has not previously been known for this class of compounds.

TABLE 9
Physical observation of patch formulation

| | Solvent | Period | | |
|---|---|---|---|---|
| Formulation | system | 6 weeks | 8 weeks | 15 weeks |
| 5.5% zolmitriptan, | Ethanol | Clear | Clear | Clear |
| 2.5% limonene, | | | | |
| 2.5% cineole | | | | |
| 5.5% zolmitriptan, | Ethanol | Clear | Clear | Clear |
| 5% cineole | | | | |

Change of Skin Permeation Flux after Storage

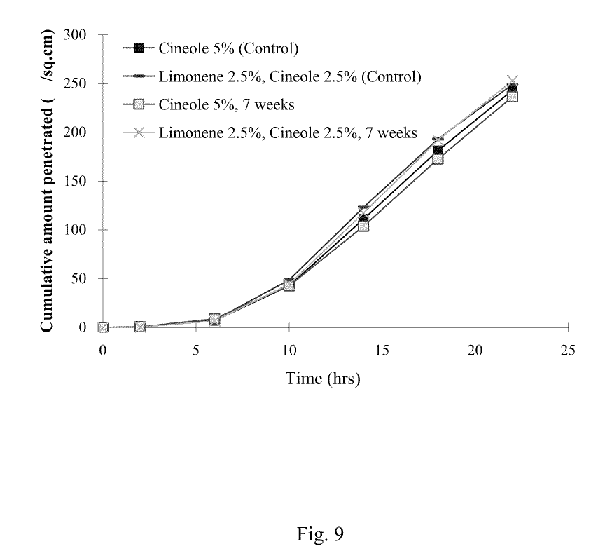

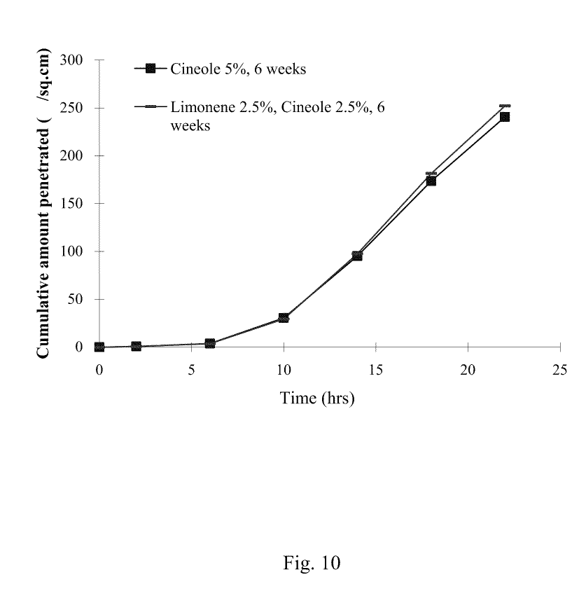

The patch stored at the room temperature for the designated period was compared with the control patch manufactured freshly for skin permeation using hairless mouse skin. The result (Table 10) showed that the cumulative skin permeation amount of each formulation was consistent regardless of storage time. No crystallization was observed and no diminution of permeation was observed. The penetration rate versus time for 24 hours is shown in FIGS. 9 and 10.

TABLE 10
Storage Stability Cumulative Permeation

| | Cumulative amount permeated (μg/sq · cm) | | |
|---|---|---|---|
| Formulation | 6 weeks | 7 weeks | 8 weeks |
| 5.5% zolmitriptan, | 252.2 | 249.2*Vs 253.2 | 271.1*Vs 264.4 |
| 2.5% limonene, | | | |
| 2.5% cineole | | | |
| 5.5% zolmitriptan, | 240.6 | 243.4*Vs 236.8 | Not measured |
| 5% cineole | | | |

*Control (Prepared fresh at the time of permeation study)

### Example 10

Formulations (18 and 20) according to Table 11 were prepared using acrylate adhesive National Adhesives Duro-Tak® 387-2516 as the PSA and ethanol as solvent for drug solution.

TABLE 11

Formulation

| | 18 | 20 |
|---|---|---|
| Components | WT % | WT % |
| Zolmitriptan | 5.5 | 5.5 |
| Cineole | 5 | 5 |
| Sucrose Ester P 1670 | | 3 |
| Flux μg/cm²hr | 16.72 | 18.44 |

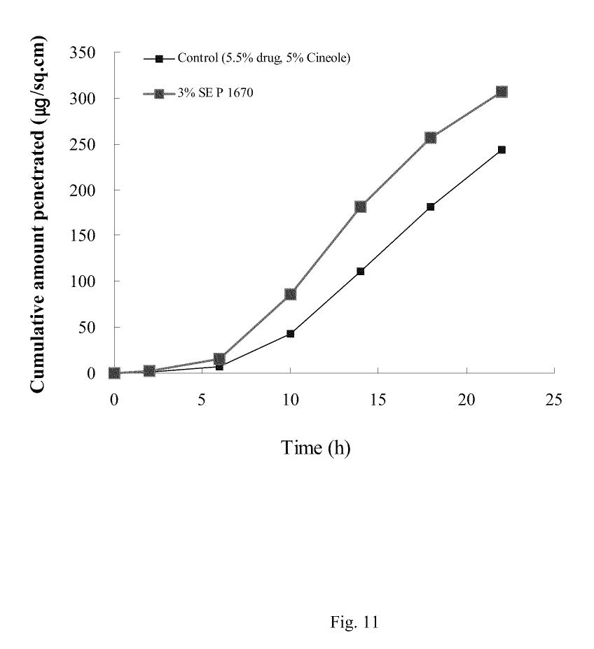

FIG. 11 shows the benefit of including cineole and sucrose ester (SE) P 1670 as enhancers in the transdermal formulation containing zolmitriptan.

TABLE 12
HLB Value of Fatty Acid Esters of Sucrose

| | Commercial | | Mono ester |
|---|---|---|---|
| Sucrose Ester | Designation | HLB value | content |
| Sucrose stearate | S-070 | <1 | <1% |
| | S-170 | 1 | 1% |
| | S-270 | 2 | 10% |
| | S-370 | 3 | 20% |
| | S-370F | 3 | 20% |
| | S-570 | 5 | 30% |
| | S-770 | 7 | 40% |
| | S-970 | 9 | 50% |
| | S-1170 | 11 | 55% |
| | S-1570 | 15 | 70% |
| | S-1670 | 16 | 75% |
| Sucrose palmitate | P-170 | 1 | 1% |
| | P-1570 | 15 | 70% |
| | P-1670 | 16 | 80% |
| Sucrose laurate | L-195 | 1 | 1% |
| | L-595 | 5 | 30% |
| | LWA-1570 | 15 | 70% |
| | L-1695 | 16 | 80% |
| Sucrose behenate | B-370 | 3 | 20% |
| Sucrose oleate | O-170 | 1 | 1% |
| | OWA-1570 | 15 | 70% |
| Sucrose erucate | ER-190 | 1 | 0% |
| | ER-290 | 2 | 2% |

The hydrophilic-lipophilic balance (HLB) of a surfactant is a measure of the degree to which it is hydrophilic or lipophilic, determined by calculating values for the different regions of the molecule, as described by W. C. Griffin “Classification of Surface-Active Agents by ‘HLB,’” Journal of the Society of Cosmetic Chemists 1 (1949): 311. and W. C. Griffin “Calculation of HLB Values of Non-Ionic Surfactants,” Journal of the Society of Cosmetic Chemists 5 (1954): 259. Other methods have been suggested, notably by J. T. Davies “A quantitative kinetic theory of emulsion type, I. Physical chemistry of the emulsifying agent,” Gas/Liquid and Liquid/Liquid Interface. Proceedings of the International Congress of Surface Activity (1957): 426-438. All three references are incorporated herein by reference. Fatty acid esters of sucrose are available from commercially, such as from Misubishi-Kagaku Foods Co.

The HLB for a combination of components with differing HLB value is by the following formula:

$$H L B for a combination of components = \frac{{\sum }_{i = l}^{n} ( {H}_{i} \times {A}_{i} )}{{\sum }_{i = 1}^{n} ( {A}_{i} )}$$

where Hᵢ is the HLB value of individual component and Aᵢ is the amount of individual component.

The fatty acid esters of sucrose may be used individually or in mixtures and in combination with other permeation enhancers. The combined fatty acid esters of sucrose will have an HLB of about 10 to about 16, preferable about 12 to about 16 and most preferably about 14 to about 16.

What is claimed is:

1. A transdermal formulation comprising:
a) an adhesive layer comprising:
i. an acrylate adhesive polymer having an —OH or no functional groups;
ii. a triptan; and
b) a backing layer.

2. The transdermal formulation of claim 1 further comprising an absorption enhancer.

3. The transdermal formulation of claim 1, wherein the triptan is present in an amount of from 0.5 wt % to 20 wt % of the weight of adhesive polymer.

4. The transdermal formulation of claim 1, wherein the triptan is zolmitriptan.

5. The transdermal formulation of claim 4, wherein zolmitriptan is present in an amount of from 0.5 wt % to 12 wt % of the weight of adhesive polymer.

6. A transdermal formulation of claim 2 wherein the absorption enhancer is a terpene.

7. The transdermal formulation of claim 6 wherein the terpene is selected from the group comprising: cineole, limonene, cymene, pinene, and pellandrene.

8. The transdermal formulation of claim 6 wherein the terpene is selected from the group comprising: cineole and limonene.

9. The transdermal formulation of claim 6 wherein the terpene is present in an amount of from about 0.5 to about 20 percent of the weight of the adhesive polymer

10. A transdermal formulation of claim 1 wherein the triptan is selected from the group comprising: eletriptan, frovatriptan, sumatriptan, zolmitriptan, naratriptan, rizatriptan, almotriptan and pharmaceutically acceptable salts thereof.

11. The transdermal formulation of claim 1 wherein said adhesive layer is acrylate polymer with hydroxyl functional group.

12. The transdermal formulation of claim 1 wherein said adhesive layer is acrylate polymer with no functional groups.

13. The transdermal formulation of claim 11, wherein the triptan is zolmitriptan and is present in an amount of from 0.5 wt % to 12 wt % of the weight of adhesive polymer.

14. The transdermal formulation of claim 12, wherein the triptan is zolmitriptan and is present in an amount of from 0.5 wt % to 12 wt % of the weight of adhesive polymer.

15. The transdermal formulation of claim 1, wherein formulation is in the form of a patch having a surface area about 5 cm² to about 140 cm².

16. The transdermal formulation of claim 1, wherein formulation is in the form of a patch having a surface area about 5 cm² to about 100 cm².

17. The transdermal formulation of claim 1, wherein formulation is in the form of a patch having a surface area about 10 cm² to about 60 cm².

18. The transdermal formulation of claim 2, wherein said permeation enhancer is selected from the group consisting of polyoxyethylene alkyl ethers such as polyoxyethylene cetyl and lauryl ethers, fatty alcohols comprising: lauryl alcohol, glycerin fatty acid ester, fatty acid alkyl esters and their derivatives, sucrose fatty acid esters, polyglyceryl-3 oleate, isopropyl myristate and isopropyl palmitate.

19. The transdermal formulation of claim 18, wherein the permeation enhancer is present in an amount of from about 0.1% weight to about 20% of the weight of the adhesive polymer.

20. The transdermal formulation of claim 6 wherein the absorption enhancer is a terpene and the triptan remains in solution in the acrylate adhesive matrix.

21. The transdermal formulation of claim 6 wherein the triptan remains unchanged in the acrylate adhesive matrix for about six months or longer.

22. The transdermal formulation of claim 6 wherein the triptan remains unchanged in the acrylate adhesive matrix for about one year or longer.

23. A method of delivering a therapeutic amount of a triptan to an individual in need thereof, the method comprising;
contacting a topical surface of said individual with a transdermal formulation comprising:
a) an adhesive layer comprising:
i. an acrylate adhesive polymer having an —OH or no functional groups;
ii. a triptan; and
b) a backing layer.

24. The method according to claim 23 wherein the triptan is selected from the group comprising: eletriptan, frovatriptan, sumatriptan, zolmitriptan, naratriptan, rizatriptan and almotriptan and pharmaceutically acceptable salts thereof.

25. The method according to claim 23 wherein the triptan is zolmitriptan.

26. The method according to claim 23, wherein said formulation is a transdermal patch and said adhesive polymer is an acrylate polymer with hydroxyl functional group.

27. The method according to claim 23, wherein said formulation is a transdermal patch and said adhesive polymer is an acrylate polymer with no functional groups.

28. The method according to claim 23 wherein said adhesive layer further comprises a permeation enhancer.

29. The method according to claim 23, wherein said method comprises adhering the transdermal patch formulation to a skin surface of an individual.

30. The method of claim 23, wherein said method is a method of treating migraine.

31. The method of claim 23, wherein the method provides for a level of triptan in the individual that is effective to inhibit migraine pain.

32. The method of claim 23, wherein the triptan is zolmitriptan provides for a therapeutic level of zolmitriptan in the individual that is effective to inhibit migraine pain.

33. The method of claim 23, wherein the triptan is zolmitriptan provides for a therapeutic level of zolmitriptan in the individual that is effective to prophylactically inhibit migraine conditions.

34. The method of claim 23, wherein the method provides a substantially constant level of triptan permeation rate into the individual over an extended period of time.

35. The method of claim 23, wherein the method provides a substantially constant level of zolmitriptan in the individual over an extended period of time.

36. The method of claim 23, wherein the transdermal formulation is applied to the patient's skin within 2 hours of the patient taking an oral or injectable triptan.

37. The method of claim 23, wherein the transdermal formulation is in the form of a patch.
